(19) United States
(12) Patent Application Publication
(10) Pub. No.: US 2011/0027791 A1
(43) Pub. Date: Feb. 3, 2011

(54) METHOD FOR GENERATING SINGLE-STRANDED DNA MOLECULES REPRESENTATIVE OF A DNA SAMPLE

(75) Inventor: Karl Frederick POETTER, Northcote (AU)

Correspondence Address: KNOBBE MARTENS OLSON & BEAR LLP, 2040 MAIN STREET, FOURTEENTH FLOOR, IRVINE, CA, 92614, US

(73) Assignee: GENERA BIOSYSTEMS LIMITED, SCORESBY VICTORIA (AU)

(21) Appl. No.: 12/882,982

(22) Filed: Sep. 15, 2010

Related U.S. Application Data
(63) Continuation of application No. 11/631,714, filed on Sep. 6, 2007.

(30) Foreign Application Priority Data
Jul. 6, 2004 (AU) 2004903706

(51) Int. Cl. C12Q 1/68

(52) U.S. Cl. 435/6

(57) ABSTRACT

A method for generating a population of single stranded DNA molecules representative of a DNA sample, including digesting the DNA sample with a Type I restriction endonuclease; subjecting the DNA fragments to melt conditions to produce a melt-generated mixture of forward and reverse single stranded DNA fragments; contacting the mixture of forward and reverse single strand DNA fragments with a solid support having immobilized thereon the complement of either the forward or reverse strand, wherein the solid phase hybridizations are favored over self-annealing of the melt-generated single stranded DNA molecules; and releasing the captured forward or reverse single strand DNA fragments from the complement immobilized to the solid support to generate a population of single stranded DNA molecules representative of the DNA sample.

### CROSS-REFERENCE TO RELATED APPLICATIONS

This application is a continuation of application Ser. No. 11/631,714, filed Sep. 6, 2007, which is the United States National Phase under 35 U.S.C. §371 of International Application PCT/AU2005/000991, filed Jul. 6, 2005 designating the U.S., and published in English as WO 2006/002491 on Jan. 12, 2006, which claims priority to Australian Patent Application No. 2004903706, filed Jul. 6, 2004.

### BACKGROUND OF THE INVENTION

1. Field of the Invention

The present invention provides a method for detecting aneuploidy in a subject. This method has applications for the detection of aneuploidy in single cells, embryos and complete organisms. The present invention has particular application for the detection of aneuploidy in human and other animal embryos generated by in-vitro fertilization. Pre-implantation screening for aneuploidy has the potential to significantly increase the rate of successful carriage to term after IVF treatment, and significantly reduce the incidence of birth defects in children conceived with the assistance of IVF treatment. Kits for the detection of aneuploidy are also provided.

2. Description of the Related Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Under normal circumstances in a diploid organism, one chromosome from each parent is transmitted to the offspring embryo. However, non-disjunction events, on the maternal, paternal or both sides can lead to embryos with aberrant chromosome number, a condition known as aneuploidy.

Euploidy is the condition of having the correct number of structurally normal chromosomes. For example, euploid human females have 46 chromosomes (44 autosomes and two X chromosomes), whereas euploid bulls have 60 chromosomes (58 autosomes plus an X and a Y chromosome).

Aneuploidy is the condition of having less than or more than the natural diploid number of chromosomes, and is the most frequently observed type of cytogenetic abnormality. In other words, it is any deviation from euploidy, although many authors restrict use of this term to conditions in which only a small number of chromosomes are missing or added.

Generally, aneuploidy is recognized as a small deviation from euploidy for the simple reason that major deviations are rarely compatible with survival, and such individuals usually die prenatally.

The two most commonly observed forms of aneuploidy are monosomy and trisomy.

Monosomy is lack of one of a pair of chromosomes. An individual having only one chromosome 6 is said to have monosomy 6. A common monosomy seen in many species is X chromosome monosomy, also known as Turner's syndrome in humans. Monosomy is most commonly lethal during prenatal development.

Trisomy is having three chromosomes of a particular type. A common autosomal trisomy in humans is Down syndrome, or trisomy 21, in which a person has three instead of the normal two chromosome 21's. Trisomy is a specific instance of polysomy, a more general term that indicates having more than two of any given chromosome (in diploid organisms).

Another type of aneuploidy is triploidy. A triploid individual has three of every chromosome, that is, three haploid sets of chromosomes. A triploid human would have 69 chromosomes (3 haploid sets of 23), and a triploid dog would have 117 chromosomes. Production of triploids seems to be relatively common and can occur by, for example, fertilization by two sperm. However, birth of a live triploid is extraordinarily rare and such individuals are quite abnormal. The rare triploid that survives for more than a few hours after birth is almost certainly a mosaic, having a large proportion of diploid cells. A chromosome deletion occurs when the chromosome breaks and a piece is lost. This of course involves loss of genetic information and results in what could be considered “partial monosomy” for that chromosome.

A related abnormality is a chromosome inversion. In this case, a break or breaks occur and that fragment of chromosome is inverted and rejoined rather than being lost. Inversions are thus rearrangements that do not involve loss of genetic material and, unless the breakpoints disrupt an important gene, individuals carrying inversions have a normal phenotype.

In a monosomic sample, with 2n−1 chromosomes, one entire chromosome and all its loci are lost. Similarly, in a 2n+1 trisomic sample, one extra chromosome is present in each cell, meaning one specific chromosome is represented three times due to a non-dysjunction event, usually in the female gametogenesis. A similar, but more pronounced situation, occurs in the case of a triploid sample in which each chromosome is represented three times instead of twice in each cell.

Pregnancies can be established in infertile women using the technique of in-vitro fertilization (IVF). In spite of the high rate of fertilization in-vitro, the rate of pregnancy following these procedures is relatively low, ranging from 15% to 25%. Cytogenetic studies of human oocytes fixed after failing to fertilize in-vitro display a relatively high incidence of chromosomal abnormalities (aneuploidy). Also, studies of many spontaneous abortions and pre-term embryos show that chromosomal abnormalities may be the main cause of fetal loss. The frequency of chromosomal abnormality in embryos generated using IVF is much higher than total abnormalities reported for sperm and oocytes.

In the IVF procedure, aneuploidy is the most frequently observed abnormality in the embryos generated. Many reports strongly indicate that chromosomal aneuploidy is the prime cause of fertilization failure in oocytes and implantation failure of embryos. Aneuploidy mainly arises during meiotic non-dysjunction; but many environmental factors may also disrupt spindle function and eventually lead to the formation of aneuploid embryos. Using methods currently known in the art to assess the embryo's gross chromosome makeup, one would perform cytogenetic analyses, such as karyotyping. However, this method is not a practical solution for single cells, and therefore cannot be performed as a pre-implantation screen.

Therefore, there is a need to develop rapid, inexpensive, automatable methods for detecting aneuploidy in an embryo that can be applied in the pre-implantation setting for in-vitro fertilization. Success rates of IVF could be increased if those embryos with aberrant chromosome numbers (aneuploid) could be screened out by a pre-implantation scan of the embryogenic genetic component.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer.

The present invention relates generally to a method for detecting aneuploidy in a subject, wherein a nucleic acid, which is representative of chromosome number the subject, is labeled with a reporter molecule. Furthermore, a non-aneuploid standard, equivalent in terms of binding specificity and amount, to the nucleic acid sample of the subject is labeled with a different reporter molecule. The sample and standard are subsequently competitively bound to a limiting amount of binding agent specific for the nucleic acid of the sample and standard. Aneuploidy is detected in the sample by an unequal binding of the sample and standard to the binding agent. This method has particular application inter alia for the detection of aneuploid embryos generated with in-vitro fertilization techniques. The present invention represents and improvement over existing methods for aneuploidy detection in animal embryos, as it is more rapid and relatively inexpensive and allows the detection of aneuploidy in human embryos prior to implantation.

For the purposes of the present invention aneuploidy is to be understood as any deviation from a euploid state in an organism, wherein euploidy is defined as a normal 2n set of chromosomes. For example a euploid human comprises a 2n number of chromosomes of 46. AU conditions that deviate from this state are considered aneuploid for the purposes of the present invention. Exemplary aneuploid conditions in humans include monosomy and trisomy wherein a given chromosome is represented by one or three copies, respectively, instead of two copies as in the euploid state. Furthermore aneuploidy in humans may be manifest as polyploidy wherein one (triploidy) or two (tetraploidy) complete sets of chromosomes are present in addition to the euploid complement of two. The present invention is predicated in part on the premise that if sampling equal amounts of DNA from each chromosome in a DNA sample, the relative contribution of each chromosome to the total DNA sample would be equal to 1/n of the total DNA, wherein n equals the number of chromosomes carried by the healthy diploid form of the organism. For example, in a non-aneuploid human subject, each chromosome would contribute 1/23 of the total DNA in a given DNA sample. However, in a monosomic sample, the relative amount of DNA from that chromosome would represent 1/45 of the total DNA, while a trisomic chromosome would represent 2/22 of the total DNA.

Therefore, if a given amount of DNA from a known control diploid DNA is competed against a like amount of DNA from a given biological sample for a limiting number of binding targets, the DNA's should bind to the targets in their relative frequencies.

The present invention relates to a method of detecting aneuploidy in a patient wherein chromosome number is represented by a nucleic acid sequence. Any nucleic acid sequence that is unique and representative of a given chromosome may be suitable for the methods of the present invention.

The present invention provides, therefore, a method for detecting aneuploidy in a subject, said method comprising:
(i) producing a reporter molecule-labeled polynucleotide sample that is representative of the abundance of a given chromosome in said subject;
(ii) producing an equivalent, non-aneuploid polynucleotide standard, labeled with a different reporter molecule;
(iii) mixing said sample and said standard with a limiting amount of binding agent, wherein said binding agent comprises an immobilized polynucleotide that is complementary to said nucleic acids;

wherein aneuploidy is detected as non-equal binding of said sample and said standard to said binding agent.

In order to detect aneuploidy in an organism, the method present invention is based on competitive binding, to a limiting amount of DNA binding agent, equal amounts of DNA from a sample and a standard of the same organism. Therefore, the method of the present invention has application to the detection of aneuploidy in any organism. Many organisms have multiple copies of their chromosomes, and the present invention has application to detect aneuploidy in any organism that normally carries single or multiple copies of a chromosome.

In a preferred embodiment of the present invention, the organism is preferably a diploid animal. In an even more preferred embodiment, the animal is a mammal such as a human or a livestock animal. In a most preferred embodiment of the present invention, the organism is a human.

In an even further preferred embodiment of the present invention, the subject is a human embryo generated using in-vitro fertilization.

The method of the present invention is able to detect aneuploidy in DNA extracted and/or amplified from a single cell. Therefore the method of the present invention is suitable inter alia for the detection of aneuploidy in human embryos generated using in-vitro fertilization, prior to implantation of the embryo.

Accordingly, the present invention contemplates a method for the detection of aneuploidy in a human embryo generated via in-vitro fertilization, prior to implantation of the embryo.

The present invention provides a method for detecting aneuploidy in a reproductive cell (gamete) of a subject, said method comprising:

(i) producing a reporter molecule-labeled polynucleotide sample that is representative of the abundance of a given chromosome in said reproductive cell;

(ii) producing an equivalent, non-aneuploid polynucleotide standard, labeled with a different reporter molecule;

(iii) mixing said sample and standard with a limiting amount of binding agent, wherein said binding agent comprises an immobilized polynucleotide that is complementary to said nucleic acids;

wherein aneuploidy is detected as non-equal binding of said sample and said standard to said binding agent.

Conveniently, the reporter molecule is a fluorescent marker or label.

In a preferred embodiment of the present invention the binding agent comprises a polynucleotide complementary to the polynucleotide of the sample and standard, wherein the binding agent polynucleotide is immobilized to a substrate, wherein the binding agent is compatible with flow cytometry.

The polynucleotide sequence of the binding agent is a polynucleotide sequence that is complementary to the nucleic acid sequence of the sample and standard, as described supra. Substrates suitable for the immobilization of the polynucleotide include, but are not limited to, membranes, slides, microspheres, microparticles and the like.

In a more preferred embodiment, the binding agent comprises a polynucleotide immobilized to a microparticle. In an even more preferred embodiment the microparticle is a silica microparticle. In a yet more preferred embodiment the silica microparticle is silanized for the covalent attachment of a nucleic acid.

In a further preferred embodiment, the binding of the labeled sample and/or standard to the binding agent, and/or relative amounts of labeled sample to standard on the binding agent, are determined using a flow cytometer. However, any detection system compatible with the reporter molecule is contemplated by the present invention.

A list of abbreviations used herein is provided in Table 1.

TABLE 1
Abbreviations

| Abbreviation | Description |
|---|---|
| IVF | In-vitro Fertilization |
| PCR | Polymerase Chain Reaction |
| 2n | The normal, euploid, number of chromosomes in a diploid |
| | organism |
| hCG | Human chorionic gonadotropin |

### BRIEF DESCRIPTION OF THE DRAWINGS

FIG. 1 is a graphical representation of a flow-cytometry dot-plot showing the relative fluorescence intensities of 2:1, 1:1 and 1:2 ratios of differentially labeled (Cy5 and fluorescein) PCR products after competitive binding to immobilized complementary DNA on microspheres. Color versions of this figure are available from the patentee upon request.

FIG. 2 is a graphical representation of a flow-cytometry dot-plot showing the relative fluorescence intensities of 2:1, 1:1 and 1:2 ratios of differentially labeled (Cy5 and fluorescein) PCR products after competitive binding to immobilized complementary DNA on microspheres in the presence of a 15 fold excess of non-complementary human DNA. Color versions of this figure are available from the patentee upon request.

FIG. 3 is a diagrammatic representation illustrating the possible configuration of an automated AmpaSand (Trade mark) silica bead based aneuploid screen. Parental DNAs are competed against each other for limiting binding sites on an immobilized sample DNA. Relative excess or deficit of either parent's alleles in the embryo due to aneuploidy us evidenced by a fluorescent shift. Color versions of this figure are available from the patentee upon request.

FIG. 4A and FIG. 4B: Generation of DNA probes for use in distinguishing specific chromosomes. 4A: is a schematic representation of a double stranded DNA molecule with 5′ and 3′ overhangs. 4B: is a schematic representation of a single stranded DNA sequence after clearage by Lambda Exo.

FIG. 5: is a schematic representation of a sequence specific single stranded DNA which can be used for identification of species specific DNA.

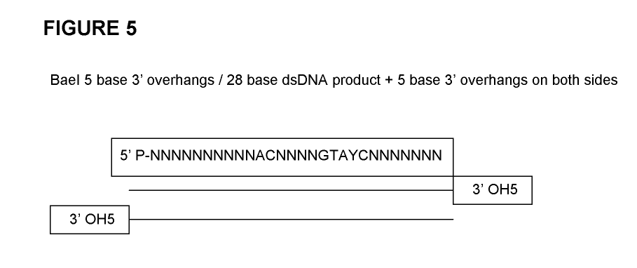

### DETAILED DESCRIPTION OF THE PREFERRED EMBODIMENT

The present invention provides a method for detecting aneuploidy in a subject. This method has application for the detection of aneuploidy in single cells, embryos and complete organisms. The present invention has particular application for the detection of aneuploidy in human and other animal embryos generated by in-vitro fertilization. Pre-implantation screening for aneuploidy has the potential to significantly increase the rate of successful carriage to term after IVF treatment, and significantly reduce the incidence of birth defects in children conceived with the assistance of IVF treatment.

Before describing the present invention in detail, it is to be understood that unless otherwise indicated, the subject invention is not limited to specific formulations of agents, manufacturing methods, methodologies, or the like, as such may vary. It is also to be understood that the terminology used herein is for the purpose of describing particular embodiments only and is not intended to be limiting.

As used in the subject specification, the singular forms “a”, “an” and “the” include plural aspects unless the context clearly dictates otherwise. Thus, for example, reference to “a binding agent” includes a single agent, as well as two or more binding agents; “on embryo” includes a single embryo as well as two or more embryos.

In describing and claiming the present invention, the following terminology is used in accordance with the definitions set forth below.

“Subject” as used herein refers to an animal, preferably a mammal and more preferably a primate including a lower primate and even more preferably, a human who can benefit from the method for detecting aneuploidy of the present invention. The subject may also be a non-animal such as a plant. A subject regardless of whether a human or non-human animal or embryo may be referred to as an individual, patient, animal, host or recipient. The methods of the present invention have applications in human medicine, veterinary medicine as well as in general, domestic or wild animal husbandry. The instant method also has application in the horticultural industry. For convenience, an “animal” specifically includes livestock species such as cattle, horses, sheep, pigs, goats and donkeys. With respect to “horses”, these include horses used in the racing industry as well as those used recreationally or in the livestock industry.

A human is the most preferred target. However, the method of the present invention is suitable for the detection of aneuploidy in any other non-human animal including laboratory test animals.

Examples of laboratory test animals include mice, rats, rabbits, guinea pigs and hamsters. Rabbits and rodent animals, such as rats and mice, provide a convenient test system or animal model as do primates and lower primates. Non-mammalian animals such as avian species, zebrafish, amphibians (including cane toads) and Drosophila species such as Drosophila melanogaster are also contemplated.

In addition, for the purposes of the present invention, the term “subject” includes all born and unborn states of the organism in question. For example, with regard to humans, “subject” as used in this specification includes all pre-natal forms of a human including the zygote, blastocyst, embryo and fetus in addition to a post natal human. This term should also be understood to encompass zygotes, blastocysts and embryos of an organism generated and/or grown in-vitro, such as embryos generated as part of an in-vitro fertilization technique. Accordingly, all pre-natal forms and in-vitro embryos for other organisms are encompassed by the methods of the present invention. A “subject” may also be a plant species.

For the purposes of the present invention aneuploidy is to be understood as any deviation from a euploid state in an organism, wherein euploidy is defined as a normal 2n set of chromosomes. For example, in a human a normal, euploid 2n number of chromosomes is 46. All conditions that deviate from this state are considered aneuploid for the purposes of the present invention. Exemplary aneuploid conditions in humans include monosomy and trisomy wherein a given chromosome is represented by one or three copies, respectively, instead of two copies as in the euploid state. Furthermore, aneuploidy in humans may be manifest as polyploidy wherein one (triploidy) or two (tetraploidy) complete sets of chromosomes are present in addition to the euploid complement of two.

In addition, for the purposes of the present invention, the term ‘aneuploidy’ should also be understood to incorporate partial monosomy conditions wherein a part of a chromosome is deleted.

The present invention is predicated in part on the premise that if sampling equal amounts of DNA from each chromosome in a DNA sample, the relative contribution of each chromosome to the total DNA sample would be equal to 1/n of the total DNA, wherein n equals the number of chromosome pairs carried by the healthy diploid form of the organism. For example, in a non-aneuploid human subject each chromosome would contribute 1/23 of the total DNA in a given DNA sample. However, in a monosomic sample, the relative amount of DNA from that chromosome would represent 1/46 of the total DNA, while a trisomic chromosome would represent 2/23 of the total DNA.

Therefore, if a given amount of DNA from a known control diploid DNA is competed against a like amount of DNA from a given biological sample for a limiting number of binding targets, the DNA's should bind to the targets in their relative frequencies.

The present invention relates to a method of detecting aneuploidy in a patient wherein chromosome number is represented by a nucleic acid sequence, referred to herein as a “sample”, “DNA sample” or “polynucleotide sample”. Any nucleic acid sequence that is unique and representative of a given chromosome may be suitable for the methods of the present invention. A person of skill in the art will be able to determine whether a given nucleic acid sequence is unique and representative for a given chromosome.

Chromosome specific polynucleotide samples suitable for the present invention may be generated by any convenient means. Exemplary methods that in no way limit the present invention include: isolation of chromosome specific polynucleotides from enzymatically or physically digested genomic DNA; amplification of chromosome specific polynucleotide sequences using PCR from genomic DNA; and identification of chromosome specific sequences via cloning and screening from genomic DNA.

Genomic DNA, suitable for the generation or identification of these chromosome specific polynucleotide samples, may be isolated using methods commonly used by those of skill in the art. The tissue used for the isolation of the genomic DNA is dependent on the particular application of the method. For example, to test for aneuploidy in a post-natal organism, somatic cells of the organism are suitable for the isolation of genomic DNA used to generate a sample according to the present invention. Alternatively, to detect non-dysjunction events in reproductive cells, the DNA from the gametes of a given organism would need to be used for the generation of the sample. Finally, to screen for aneuploidy in a prenatal embryo, a blastomere would be the most appropriate tissue from which to generate the sample.

For the purposes of the present invention a ‘standard’ is to be understood as an equivalent nucleic acid to the sample, but wherein the standard is generated from the genomic DNA of a known, non-aneuploid source. Therefore, in the case of a diploid organism, it is known that each chromosome is represented twice in the standard.

The term ‘equivalent’ with regard to the sample and standard, is to be understood as equal binding to a given nucleic acid sequence, such as is part of the binding agent of the present invention, under the conditions used for hybridisation. For example, under very high stringency conditions, the nucleic acid sample, standard and binding agent may all have to have 100% identical polynucleotide sequences for equal binding of the sample and standard to the binding agent. However, at lower stringency, the sample and standard may have somewhat different polynucleotide sequences to each other, yet have equal binding affinity for the polynucleotide of the binding agent. Therefore, it is possible for one skilled in the art to determine what constitutes equivalency with regard to the standard and sample when hybridization conditions are considered. However, it is preferred that the sample and standard comprise identical polynucleotide sequences, and the binding agent comprises a polynucleotide sequence complementary to the sample and standard.

Partial loss of a given chromosome, known as deletion or partial monoploidy, may be detected using the method of the present invention when the sample of the chromosome is chosen from within a potentially deleted region. Furthermore, partial deletions may be confirmed by application of the method of the present invention using a marker within a putatively deleted region in comparison to a marker on the same chromosome outside the putatively deleted region. In this situation, a partial deletion of the chromosome would be detected as monoploidy using one marker on the chromosome and diploidy using another marker on the same chromosome.

The present invention further contemplates the labeling of a nucleic acid that is representative of a chromosome with a reporter molecule such as a fluorescent marker. Many different fluorescent markers will be familiar to those of skill in the art, and the choice of fluorescent marker in no way limits the subject invention. In a preferred embodiment, the fluorescent markers of the present invention comprise any fluorescent marker that can be attached to a polynucleotide and is excitable using a light source selected from the group below:
(i) Argon ion lasers—comprise a blue, 488 run line, which is suitable for the excitation of many dyes and fluorochromes that fluoresce in the green to red region. Tunable argon lasers are also available that emit at a range of wavelengths (458 nm, 488 nm, 496 nm, 515 nm and others).
(ii) Diode lasers—have an emission wavelength of 635 nm. Other diode lasers which are now available operate at 532 nm. This wavelength excites propidium iodide (PI) optimally. Blue diode lasers emitting light around 476 nm are also available.
(iii) HeNe gas lasers—operate with the red 633 nm line.
(iv) HeCd lasers—operate at 325 nm.
(v) 100 W mercury arc lamp—the most efficient light source for excitation of UV dyes like Hoechst and DAPI.

In more preferred embodiments of the present invention the fluorescent markers are selected from: Alexa Fluor dyes; BoDipy dyes, including BoDipy 630/650 and BoDipy 650/665; Cy dyes, particulary Cy3, Cy5 and Cy 5.5; 6-FAM (Fluorescein); Fluorescein dT; Hexachlorofluorescein (Hex); 6-carboxy-4′, 5′-dichloro-2′, T-dimethoxyfluorescein (JOE); Oregon green dyes, including 488-X and 514; Rhodamine dyes, including Rhodamine Green, Rhodamine Red and ROX; Carboxytetramethylrhodamine (TAMRA); Tetrachlorofluorescein (TET); and Texas Red. In particularly preferred embodiments of the present invention, the markers are fluorescein and Cy5.

In order to differentiate the standard from the sample for the purposes of the present invention, it is preferred that the labels for the sample and the standard have distinct emission spectra.

The choice of method for the attachment of the fluorescent marker to the polynucleotide or incorporation of the marker into the polynucleotide during synthesis or amplification in no way limits the present invention. AU methods for fluorescently labeling a polynucleotide are contemplated by the present invention. Exemplary methods include both pre- and post-synthesis methods for labelling of polynucleotides. Pre-synthesis methods include labelling of a PCR primer that is subsequently used for amplification of, and thereby incorporated into, a polynucleotide via PCR. In this method, the fluorescent marker is typically attached to the 5′ end of a primer suitable for the amplification of the polynucleotide. Also a linker is typically used between the fluorophore and the polynucleotide molecule. Appropriate linker sequences will be readily ascertained by those of skill in the art, and are likely to include linkers such as C6, C7 and C12 amino modifiers and linkers comprising thiol groups. As will be readily ascertained, a primer may comprise the linker and fluorophore, or the linker alone, to which the fluorophore may be attached at a later stage. Post synthetic labeling methods include nick-labelling systems wherein a labeled polynucleotide is synthesised by Klenow polymerase from random primers. Fluorescent labeled nucleotides, or nucleotides comprising a linker group, may be incorporated into the Klenow polymerase synthesised polynucleotide during synthesis. However, it should be understood that the present invention is in no way defined or limited by the choice of labeling method.

Accordingly, the present invention provides a method for detecting aneuploidy in a subject, said method comprising:
(i) producing a reporter molecule-labeled polynucleotide sample that is representative of the abundance of a given chromosome in said subject;
(ii) producing an equivalent, non-aneuploid polynucleotide standard, labeled with a different reporter molecule;
(iii) mixing said sample and standard with a limiting amount of binding agent, wherein said binding agent comprises an immobilized polynucleotide that is complementary to said nucleic acids;

wherein aneuploidy is detected as non-equal binding of said sample and said standard to said binding agent.

Preferably, the reporter molecule is a fluorescent label.

In order to detect aneuploidy in an organism including an embryo, the method of the present invention is based on the competitive binding, to a limiting amount of complementary binding agent, of equal amounts of DNA from a sample and a standard of the same organism. Therefore, the method of the present invention has application to the detection of aneuploidy in any organism. Many organisms have multiple copies of their chromosomes, and the present invention has application to detect aneuploidy in any organism that normally carries single or multiple copies of a chromosome. Exemplary organisms include, but in no. way limit the invention: haploid organisms such as the males of certain species of wasp, bee and ant; triploid organisms such as oysters; diploid organisms such as animals, particularly humans; tetraploid organisms, including several plant species such as cyclamen and the American Elm, and some species of frog and toad; and hexaploid organisms such as the plant Triticum aestivum.

In a preferred embodiment of the present invention, the organism is diploid, and more preferably an animal. In an even more preferred embodiment, the animal is a mammal, more preferably a livestock animal or human. In a most preferred embodiment of the present invention the organism is a human. The present invention, however, extends to non-animal species such as plants.

In a further preferred embodiment of the present invention, the human subject is a human embryo generated using in-vitro fertilization.

In-vitro fertilization comprises four basic steps: ovary stimulation, egg retrieval, insemination, and embryo transfer. An example of the IVF procedure in humans is detailed below:

(i) Ovulation Induction—To stimulate the ovaries to produce more eggs, human menopausal gonadotropins are administered, which are concentrated forms of the natural hormones that stimulate ovulation. Gonadotropins cause several follicles to mature at once, ranging from two to thirty in humans. When the eggs are determined to be mature, one dose of human chorionic gonadotropin (hCG) is administered. hCG prepares the eggs for ovulation and fertilization. Here, it acts as a timekeeper indicating that approximately 40 hours from the moment of intake, ovulation will naturally occur. Therefore, egg retrieval must take place approximately 36 hours after this dose of hCG.

(ii) Egg Retrieval—A needle is placed into the ovary and fluid and eggs are removed from the follicles by a suction drive. The eggs are then placed into a test tube. On average, over two thirds of the follicles produce eggs.

(iii) Insemination and Fertilization—The eggs are allowed to mature for several hours before sperm are added, usually 6 to 8 hours after the retrieval. Insemination is simply the addition of the sperm to the culture media; each egg is isolated in its own dish and a defined number of sperm are placed with each one. The dishes are then placed in an incubator set at physiological temperature. Several hours later fertilization occurs when the sperms actually enter the egg. When this happens, the sperm loses its tail and its head enlarges. This stage is known as the 2PN stage because the two pro-nuclei have not fused yet. The embryo begins dividing, first into two and then four cells. Usually 36 to 48 hours after retrieval, the embryos cleave into four cells.

(iv) Embryo Transfer and Implantation—Embryo transfer (implantation) occurs 72 hours after egg retrieval. The embryos are drawn into a catheter and the fluid, containing the embryos, is deposited into the uterine cavity. The number of embryos transferred varies. After the transfer, it is up to the embryo to find and attach itself to the uterine wall.

In addition to assisting infertile humans reproduce, in-vitro fertilization has application in agriculture. For example in cattle, in-vitro fertilization has contributed to improvements in the genetic stock of cattle. Examples include:

(i) Older Cows—In the past, advanced age caused many cows with genetic merit to be eliminated from the breeding pool. These valuable old females may be able to generate a low-risk harvest of immature oocytes, or eggs.

(ii) Problem Cows—Females of all breeds and ages may have reproductive difficulties due to environmental causes: ovulatory failure, oviductal transport failure, disease/degeneration of the uterus, and non-responsiveness to stimulatory hormones. Even with these conditions, many cows can be managed to produce ovarian follicles which contain recoverable oocytes.

(iii) Healthy Cycling Females—Donor females can be enrolled in an in-vitro fertilization program simultaneously with the classical multiple ovulation and embryo transfer. By combining oocyte retrieval and the in-vitro fertilization program between rest periods in the superovulatory process, donors reach maximum success.

Accordingly, the method of the present invention should also be understood to encompass screening for aneuploidy in both human and non-human embryos generated using in-vitro fertilization techniques.

Current methods in the art for the detection of aneuploidy in embryos are based on post-implantation screens. Jenderney et al. (Mol. Hum. Reprod. 6(9): 855-860, 2000) describe the method of using QF-PCR, specific for short tandem repeats on specific chromosomes, on samples of amniotic fluid. It is also possible to assess potential aneuploidy in a fetus from fetal cells in the maternal blood stream, using techniques such as fluorescent in-situ hybridization (Bianchi et al, Prenat. Diag. 22(7): 609-615, 2002). However, these techniques are only suitable for the detection of aneuploidy in an embryo or fetus post-implantation.

The method of the present invention is able to detect aneuploidy in DNA extracted and/or amplified from a single cell. Therefore, the method of the present application is suitable, inter alia, for the detection of aneuploidy in animal embryos generated using in-vitro fertilization, prior to implantation of the embryo.

Single cells may be isolated from embryos using standard blastomere biopsy techniques, as will be known to those of skill in the art. Briefly, the blastomere biopsy procedure comprises the following steps:

(i) A 7-cell embryo, on Day 3 after IVF, is ready to be biopsied. It is held in place on a micromanipulator with a holding pipette.

(ii) A zona drilling pipette is used to drill a hole through the shell of the embryo (the zona) using acid Tyrode's.

(iii) The embryo biopsy pipette is then introduced through this opening, and gentle suction is applied to dislodge a single cell (a blastomere) from the embryo.

(iv) The biopsied embryo is then returned to the incubator for further culture. The blastomere can now be screened for aneuploidy according to the method of the present invention.

(v) Based on the analysis of the blastomere, corresponding non-aneuploid embryos are then selected for implantation.

Accordingly, the present invention provides a method for the detection of aneuploidy in an animal embryo generated via in-vitro fertilization, prior to implantation of the embryo.

In a preferred embodiment of the present invention, the animal embryo is a human embryo.

In addition to the detection of chromosome number in an organism, the present invention has application for the detection of non-dysjunction events in reproductive cells. In this aspect of the present invention, gametes of an organism, preferably a human, may be tested for missing and/or duplicated chromosomes. The method of this aspect of the present invention would be largely similar to the method described above. Briefly, a nucleic acid representative of a given chromosome in a gamete is labeled with a reporter molecule (eg., a fluorescent marker), while an equivalent representative polynucleotide from a known non-aneuploid gamete is labeled with a different fluorescent marker. As with the method described for detection of aneuploidy in a somatic or embryogenic cell, the sample and standard polynucleotides are competitively bound to a limiting number of binding agents. A missing chromosome in the sample, would be manifest as an increased detection of the standard on the binding agent. Duplication of a chromosome in the sample would be detected as an increased binding of sample to the binding agent. In the case where no non-dysjunction events have occurred in the sample, binding of the standard and sample to the binding agent should be approximately equal.

Binding agents contemplated by the present invention comprise a polynucleotide sequence immobilised to a substrate. The polynucleotide sequence of the binding agent comprises a polynucleotide sequence that is complementary to the nucleic acid sequence of the sample and standard, as described supra.

By complementary, it is to be understood that the immobilized polynucleotide of the present invention should bind to the chromosome-number representative polynucleotide of the sample and standard under low stringency conditions. Preferably, the immobilized polynucleotide should bind to the sample and standard under medium stringency conditions, and most preferable the immobilized polynucleotide should bind to the sample and standard under high stringency conditions.

Reference herein to low stringency includes and encompasses from at least about 0 to at least about 15% v/v formamide (including 1%, 2%, 3%, 4%, 5%, 6%, 7%, 8%, 9%, 10% 11%, 12%, 13% and 14% v/v formamide) and from at least about 1 M to at least about 2 M salt for hybridization, and at least about 1 M to at least about 2 M salt for washing conditions. Generally, low stringency is at from about 25-30° C. to about 50° C. such as 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide including 16% v/v, 17% v/v, 18% v/v, 19% v/v, 20% v/v, 21% v/v, 22% v/v, 23% v/v, 24% v/v, 25% v/v, 26% v/v, 27% v/v, 28% v/v, 29% v/v, 30% v/v and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide such as 31% v/v, 32% v/v, 33% v/v, 34% v/v, 35% v/v, 36% v/v, 37% v/v, 38% v/v, 39% v/v, 40% v/v, 41% v/v, 42% v/v, 43% v/v, 44% v/v, 45% v/v, 46% v/v, 47% v/v, 48% v/v, 49% v/v, 50% v/v and from at least about 0.01 M to at least about 0.15 M salt for hybridization, and at least about 0.01 M to at least about 0.15 M salt for washing conditions. In general, washing is carried out Tm=69.3+0.41 (G+C) % (Marmur and Doty, J. Mol. Biol. 5: 109, 1962). However, the Tm of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 650 C; high stringency is 0.1×SSC buffer, 0.1% w/v SDS at a temperature of at least 65° C.

Methods of immobilizing a polynucleotide to a substrate are well known to those of skill in the art. For the purposes of the present invention, the actual substrate used for the immobilization of the binding agent polynucleotide does not affect the application of the present invention. Therefore the binding agent of the present invention encompasses a polynucleotide immobilized onto any substrate. Non-limiting examples of the immobilisation of polynucleotides on a substrate include: dipsticks; polynucleotides immobilized to membranes, including nitrocellulose and nylon, as used for Southern blotting; immobilized polynucleotides on glass or ceramic surfaces such as slides, as used in microarrays and the like; immobilized polynucleotides on bead based substrates such as microspheres which are suitable for analysis using flow cytometry.

The polynucleotide can be attached to the substrate using any convenient means, typically this is done by physical adsorption or chemical linking. In addition, substrates may be further coated with an agent that promotes or increases the adsorption or binding of the polynucleotide to the surface of the substrate, such as amino-silanes. However, other agents that perform this function will be readily identified by persons of skill in the art. In a preferred embodiment of the present invention the binding agent comprises a polynucleotide complementary to the polynucleotide of the sample and standard, wherein the binding agent polynucleotide is immobilized to a substrate, and the binding agent is compatible with flow cytometry.

Microparticles are beads and other particles, typically in a size range of 0.05 μm diameter to 1000 μm diameter inclusive such as 0.05 μm, 0.06 μm 0.07 μm, 0.08 μm, 0.09 μm, 0.1 μm or 0.1 μm, 0.2 μm, 0.3 μm, 0.4 μm, 0.5 μm, 0.6 μm, 0.7 μm, 0.8 μm, 0.9 μm, 1 μm or 1 μm, 2 μm, 3 μm, 4 μm, 5 μm, 6 μm, 7 μm, 8 μm, 9 μm, 10 μm or 10 μm, 20 μm, 30 μm, 40 μm, 50 μm, 60 μm, 70 μm, 80 μm, 90 μm, 100 μm or 100 μm, 200 μm, 300 μm, 400 μm, 500 μm, 600 μm, 700 μm, 800 μm, 900 μm, 1000 μm. The material of the particle is commonly a compound selected from: glass, silica, alginate, gelatine, agar, cellulose, chitosan, poly-lactic acid, poly D,L-lactice-co-glycolic acid (PLGA), polystyrene, pylymethylmethacrylate (PMMA), melamine and gold. However, the present invention is not limited to microparticles of these materials, as any material to which a polynucleotide may be adsorbed, covalently bound, or otherwise attached, is contemplated by the present invention.

Polynucleotides may be encapsulated in microparticles during their production or may be attached to their surface post-production. The choice method used to associate the polynucleotide with the substrate will depend on the material used, as would be readily ascertained by the skilled artisan. In addition, further treatments, including silanization (coating of the substrate with silanes), may be performed on the microparticles prior to attachment of the polynucleotide in order to increase the binding of said polynucleotide to the microparticle.

Generally, microparticles may be coated with any compound that will covalently attach, or otherwise adsorb, to the surface of the microparticle, and in addition the agent should also have a chemical moiety for the attachment of a polynucleotide, such as a thiol, amine or carboxyl group. Examples of compounds with these characteristics include amino-terminated silanes such as amino-propyltrimethoxysilane or amino-propyltriethoxysilane. In addition to silanes, compounds such as poly-L-lysine that non-covalently attach to the glass surface and electrostatically adsorb the phosphate groups of the polynucleotide are also within the scope of the present invention. Therefore, other compounds, including other silanes suitable for the attachment of a polynucleotide to a surface would be readily identified by the skilled artisan, and the present invention is not limited by the choice of compound.

In a more preferred embodiment, the binding agent comprises a polynucleotide immobilized to a microparticle. In an even more preferred embodiment said microparticle is a silica microparticle. In a yet more preferred embodiment the silica microparticle is silanized for the covalent attachment of a nucleic acid.

The detection of fluorescent compounds via excitation with a light source and detection at a specific wavelength can be applied to a variety of instruments. Specific light sources and photodetectors have been applied to microscopes for the techniques of epifluorescence microscopy and confocal laser microscopy. Flow cytometry also uses a fluorescence based detection system for cell sorting. In addition, a number of specialized detection apparatus have been developed for the purposes of assessing fluorescence for particular applications such as microarray readers. The method of the present invention is not defined by the method and/or apparatus used for the detection of the fluorescent labels. The apparatus for detection will depend on the substrate to which the binding agent is attached. For example, binding agents comprising microparticles would likely be compatible with a flow cytometry based detection system, whereas a binding agent comprising a nucleic acid immobilized to a slide would likely be analysed using epifluorescence or laser scanning confocal microscopy. Finally, a number of binding agents arranged in an array on a slide would most likely be analysed using a specialized array reading apparatus. As can be ascertained from the above, the choice of detection method for the binding agent and bound labeled nucleic acid does not define or limit the present invention in any way, and is merely a function of the method of immobilization used for the binding agent.

However, in a further preferred embodiment of the present invention, the binding of the labeled sample and/or standard to the binding agent and/or the detection of the relative amount of labeled sample to standard bound to the binding agent are determined using a flow cytometer.

The present invention further provides a kit useful in detecting aneuploidy in organism, embryo or reproductive tissue. The kit is conveniently in a multi-compartment form wherein a first compartment comprises a reporter molecule labeled such as a fluorescently labeled oligonucleotide primer set suitable for the amplification of a chromosome specific genomic DNA sequence. A second compartment comprises the oligonucleotide primers with identical sequence to the first compartment, but with a different reporter molecule. In a third compartment is a binding agent comprising a polynucleotide sequence complementary to the predicted amplicon of the oligonucleotide primers, that is immobilzed to a substrate, such as but not limited to a microparticle. In addition to these components, instructions for the use of the kit may also be included. It is not a requirement that the kit be in multi-compartment form and it is possible to combine the contents of two or more of the compartments.

The present invention is further described by the following non-limiting examples:

### Example 1

### Sensitivity of Fluorescent Detection

The development of a high speed, low-cost, aneuploid scan is contingent first on the sensitivity of the system to discriminate input fluorescent ratios of 2:1, 1:1, and 1:2 in respect of DNA sequences labelled with Cy5 or fluorescein.

To test this capacity, PCR products from 24 human samples were pooled. 200 ng of DNA from the pool was incubated with saturating amounts of Cy5 probe or saturating amounts of fluorescein probe. The DNA was incubated at 990 C for 2 minutes followed by 10 minutes at RT. Probed PCR products were then mixed, in the ratios below (Table 2), with approximately 1,000 AmpaSand™ Beads with immobilized targets specific for the PCR product:

TABLE 2
Ratio testing for DNA content

| | Cy5 labeled PCR | Fluorescein labeled |
|---|---|---|
| Tube | product (ng) | PCR product (ng) |
| 1 | 20 | 0 |
| 2 | 0 | 20 |
| 3 | 10 | 5 |
| 4 | 10 | 10 |
| 5 | 5 | 10 |

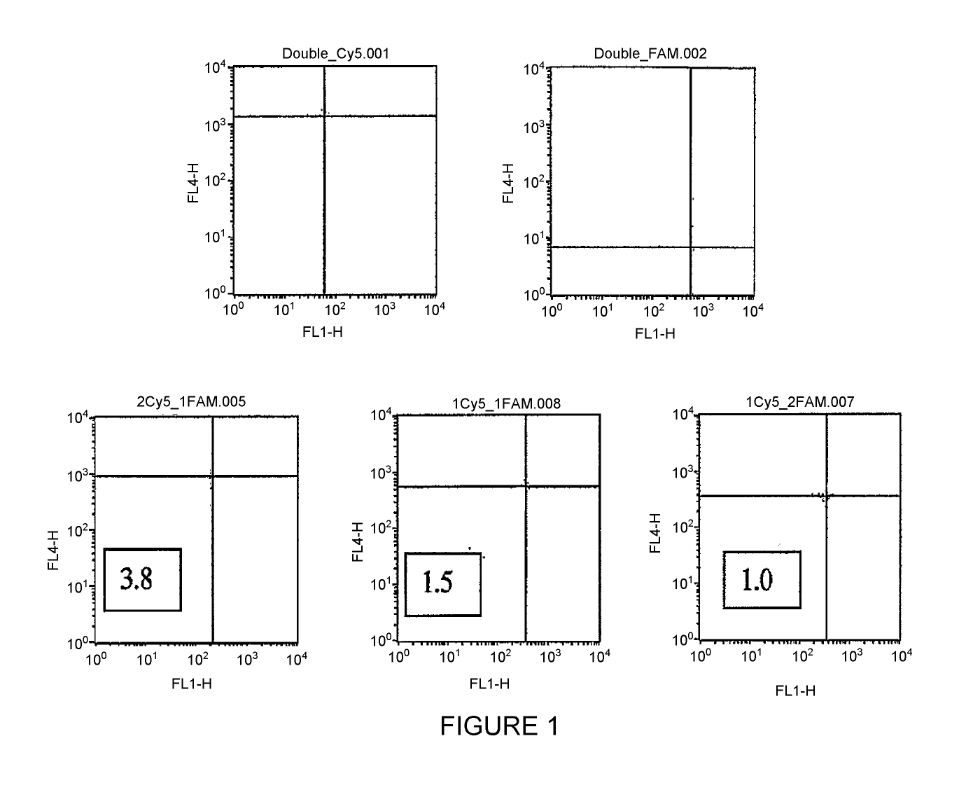

Samples were brought to 200 μl and then the beads were analysed on a BD FACSCalibur with two lasers. The dot plots are shown in FIG. 1.

The results illustrated in FIG. 1 demonstrated that the fluorescence-based method was sufficiently sensitive for the discrimination of 2:1, 1:1 and 1:2 labeled DNA ratios, and justified the next step. For the proposed scheme to work, the beads targeting each chromosome specific sequence must be able to discriminate these ratios in a background of approximately 20 fold excess of non-complementary DNA. To test if this sensitivity is achievable, the mock-up as described below was performed.

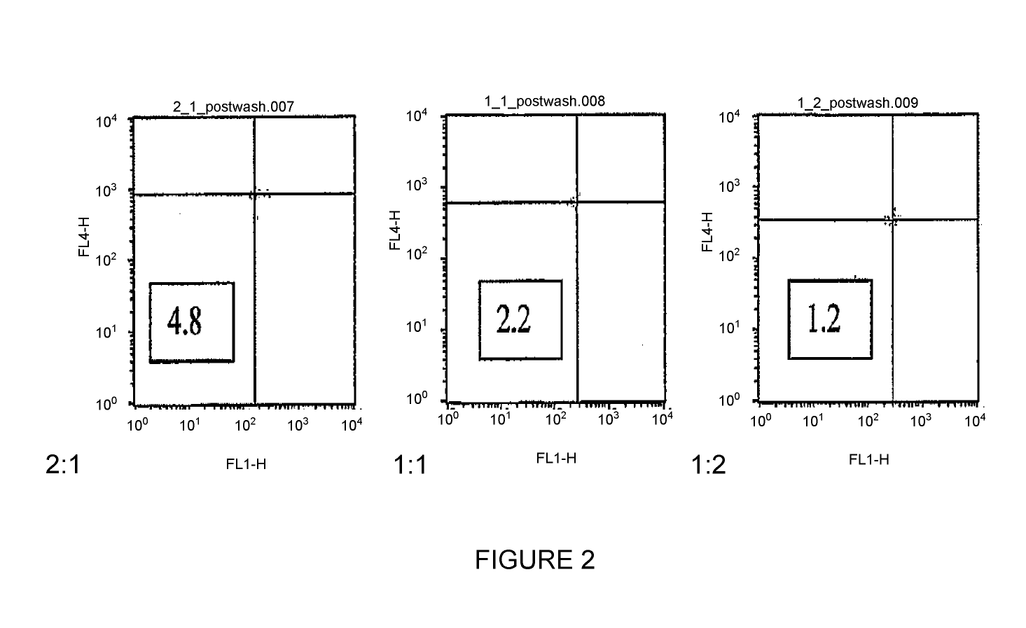

The same DNAs as used in the preceding experiment were mixed in the same ratios, but in the context of 15 fold excess of a human PCR product (approx. 220 ng of non-hybridizing PCR product) with no complementarity to the bead immobilized target. After 99° C. for two minutes, hybridisation was performed for 35 minutes at RT. The beads were read and the results are shown in FIG. 2. The ratio of Cy5 fluorescein is shown to the left of each dot plot (Cy5 level on left: Fluorescein on right) and the fluorescence ratio is given in a box within the dot plot.

These data suggest that the discrimination is actually improved in the context of large amounts of irrelevant DNA. This could be due to the longer hybridisation time, or the fact that a wash step was added to reduce the volume before cytometric analysis. Alternatively, the method could be robust and the sensitivity levels repeatable and adequate for the application.

### Example 2

### Fluorescence Based Method to Detect Aneuploidy in an Embryo

This approach has several advantages over methods currently used for the detection of aneuploidy in embryos. First, while the technique requires the same reagents for generation of fluorescently labeled PCR products, the analysis is completely automated from DNA generation to final analysis. Second, the running costs are minimal because the reader for the output is a standard flow cytometer, which is currently available in most laboratories, especially Pathology laboratories, worldwide. Third, the method works equally well in non-dysjunctions during 1st or 2nd meiosis in either parent. Fourth, the platform is microsphere-based and compatible with currently available flow cytometers as well as possible easy direct transferal to microsphere based platforms currently in development.

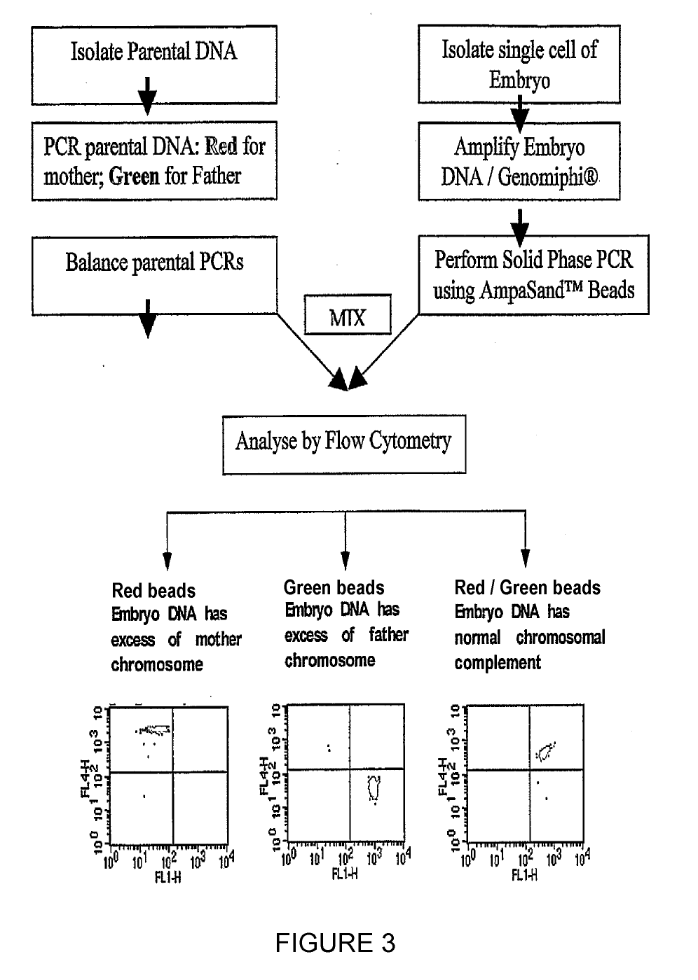

The present invention comprises the use of the parents as normal controls and the DNA from the embryo as the unknown sample in a competitive hybridisation scheme in which relative fluorescence shifts detected on microspheres in flow cytometry are used to indicate allele number discrepancies between sample and controls (FIG. 3). The major advantages of this scheme over a locus-by-locus sizing approach are substantial and include:

(i) It is possible to perform all the hybridizations and readouts in one tube, with no wash steps, using homogeneous conditions for all loci.

(ii) The analysis time for each embryo is completely automated, and performed in less than 1 minute per embryo using a standard flow cytometer.

### Example 3

### Generation of Probes

In order to determine the number of specific chromosomes in a sample, beads can be conjugated with DNA that has been derived from one particular chromosome. The beads can then be added to the sample and the relative intensity of staining analysed, thereby demonstrating the presence of one, two or more chromosomes.

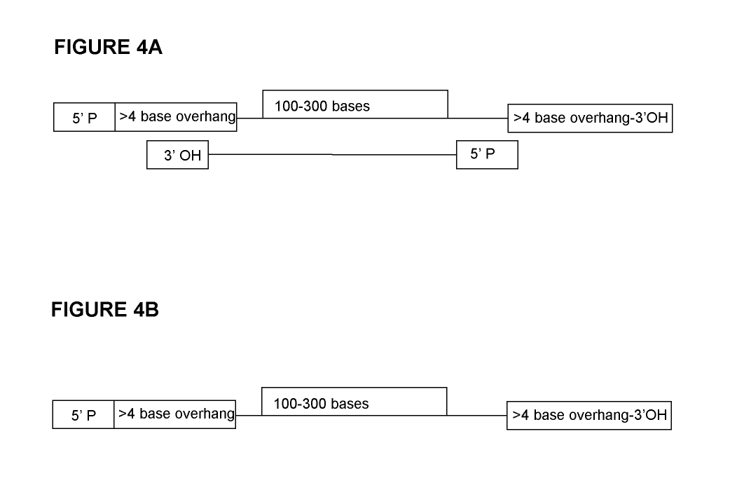

Probes are generated using Lambda Exo. Lambda Exo is a double stranded DNA exonuclease which degrades double stranded DNA in a 5′ to 3′ direction. The 5′ end must be double stranded and phosphorylated. This enzymatic reaction can be used to preferentially degrade specific strands of double stranded DNA. For making ssDNA specific targets for chromosome specific beads, a collection of DBA regions is made as shown in FIG. 4a. This includes a 5′ overhang of >4 bases, an intervening region of between 100-300 bases and a >4 base overhang at the 3′-end.

The 5′ overhang can be produced by either a Type I or Type II Restriction endonuclease, examples of which include:

TABLE 3
Restriction enzymes suitable for a 5′ overhang

| Enzyme | Overhang Length | Sequence |
|---|---|---|
| BamHI | 4 | GATC |
| EcoRI | 4 | AATT |
| HindIII | 4 | AGCT |
| AflII | 4 | TTAA |
| AgeI | 4 | CCGG |
| ApaLI | 4 | TGCA |
| ApoI | 4 | AATT |
| BanI | 4 | Variable |
| BclI | 4 | GATC |
| BglII | 4 | GATC |
| BsaI | 4 | Variable |
| BsaJI | 4 | Variable |
| BsaWI | 4 | GGCC |
| BseYI | 4 | CCAG |
| BsiWI | 4 | GTAC |
| BsmAI | 4 | Variable |
| BsmBI | 4 | Variable |
| BsmFI | 4 | Variable |
| BsoBI | 4 | Variable |
| BspEI | 4 | CCGG |
| BshHI | 4 | CATG |
| BspMI | 4 | Variable |
| BsrFI | 4 | CCGG |
| BsrGI | 4 | GTAC |
| BssHII | 4 | CGCG |
| BssKI | 4 | CCNG |
| BssSI | 4 | TCGT |
| BstEII | 4 | GTNA |
| BstYI | 4 | GATC |
| BtgI | 4 | Variable |
| DpnII | 4 | GATC |
| EaeI | 4 | GGCC |
| KasI | 4 | GCGC |
| *MboI | 4 | GATC |
| MfeI | 4 | AATT |
| MluI | 4 | CGCG |
| NcoI | 4 | CATG |
| NgoM IV | 4 | CCGG |
| NheI | 4 | CTAG |
| NotI | 4 | GGCC |
| PaeR7I | 4 | TCGA |
| PspGI | 5 | Variable |
| SalI | 4 | TCGA |
| *Sau3AI | 4 | GATC |
| SexAI | 5 | Variable |
| SfcI | 4 | Variable |
| SgrAI | 4 | CCGG |
| SpeI | 4 | CTAG |
| StyI | 4 | Variable |
| TliI | 4 | TCGA |
| Tsp45I | 4 | Variable |
| *Tsp509I | 4 | AATT |
| XbaI | 4 | CTAG |
| XhoI | 4 | TCGA |
| XmaI | 4 | CCGG |

TABLE 4
Restriction enzymes suitable for a 3′ overhang

| Enzyme | Overhang | Sequence |
|---|---|---|
| AatII | 4 | ACGT |
| ApaI | 4 | CCGG |
| BanII | 4 | Variable |
| Bme1580I | 4 | Variable |
| BsiHKAI | 4 | Variable |
| Bsp1286I | 4 | Variable |
| BstXI | 4 | Variable |
| FseI | 4 | GGCC |
| HaeII | 4 | CGCG |
| Hpy99 | 4 | Variable |
| KpnI | 4 | GTAC |
| *NlaIII | 4 | GTAC |
| NsiI | 4 | ACGT |
| NspI | 4 | GTAC |
| PstI | 4 | TGCA |
| SacI | 4 | TCGA |
| SphI | 4 | GTAC |
| *TspRI | 8 | Variable |

For example, there are approximately 60,000 unique sites per mammalian chromosome which has the preferred arrangement of restriction sites, that is a 5′ overhang site, followed by less than a 300 base pair unique single copy DNA, followed by a 3′ overhang site.

After restriction by the enzyme pair, Lambda Exo nuclease digestion yields a product as shown in FIG. 4b.

### Example 4

### Microbial Fingerprinting Using Beads

There are several restriction enzymes that cut in a way to produce defined lengths of DNA (Table 5). These enzymes rely on internal recognition sequences in the DNA. The enzymes cut, on average (assuming a G+C content of 50%), about once every 2,000 bases. For example, E. coli genome size of 4.5 million bases would have approximately 2,000 different BaeI fragments of size 28, in addition to the “smear” of fragment sizes between the BaeI sites. Plasmodium falciparum, on the other hand, would have about the same number of fragments even though the genome size is 22 million. This is due to the high A+T content of the Plasmodium genome.

TABLE 5

| | | Length of |
|---|---|---|
| Enzyme | Sequence | fragment (ds/ss) |
| AloI | (7/112)GAACNNNNNNTCC(12/7) | (26/31) |
| BplI | (8/13)GAGNNNNNCTC(13/8) | (27/32) |
| BaeI | (10/15)ACNNNNGTAYC(12/7) | (28/32) |
| Fal I | (8/13)AAGNNNNNCTT(13/8) | (27/32) |
| Hin4 I | (8/13)GAYNNNNNVTC(13/8) | (27/32) |
| Ppi I | (7/12)GAACNNNNNCTC(13/8) | (27/32) |
| Psr I | (7/12)GAACNNNNNNTAC(12/7) | (27/32) |

Type I restriction enzymes for small “sticky-ended” fragment generation. Numbers in
parentheses denote leading or trailing number of Ns. First number denotes forward
strand, second number denotes reverse strand. For clarification, see FIG. 1, in which
Bae I is shown.

The production of libraries of organism specific BaeI fragments would be relatively simple. Clones could be arrayed in duplicate and probed with labelled genomic DNA from original source versus comparative genome.

Cutting a microbial or viral genome will generate a collection of 33 base fragments (internal 28 ds). Many of these fragments will be unique to a particular organism, or class of organism. Exact complementary probes can be constructed and immobilized on AmpaSand™ or Q-Sand™ Beads. By binding to the bead, the readout can be by either FRET based fluorescence or by Whispering Gallery Mode shifts. The solid phase hybridizations will be favored over self-annealing because of the perfect match over 33 contiguous bases rather than 28.

### Example 5

### Mouse Genotyping

### MuraSand™ Beads

Mouse genetics often requires a long-term breeding strategy in which a gene of one mouse strain (the donor strain) is introduced into another mouse strain, usually a standard strain denoted P1. This often requires many backcrosses and much time. One problem inherent in this strategy is that unwanted genes from the donor strain are carried along into subsequent generations. These unwanted genes, passenger loci, can be screened before matings, but at a very large cost in both money and time.

MuraSand™ Beads are a way to drastically reduce both time and money for this process. Alleles for major strains used in most genetic experiments are chosen for specific clusters in the genome. The assays for up to 20 different loci are contained on a specific bead. Probes are constructed in such a way that red labelled probes match the P1 strain and green labelled probes match the donor strain. In this way, by reading the beads from each mouse, only the mice yielding the reddest beads will be chosen for further matings.

This process is also applicable to producing new strains of mice that have multiple genomic regions of donor loci. For example, beads specific for chromosome 19 could be engineered so that only mice yielding green beads for this region, but red for all others are chosen for the next back-cross generation.

This technique is also applicable to cattle using BoviSand™ Beads, as well as other species of animals using species specific beads.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bianchi et al, Prenat. Diag. 22(7):609-615, 2002.
Bonner and Laskey, Eur. J. Biochem. 46:83, 1974.
Jenderney et al, Mol. Hum. Reprod. 6(9):855-860, 2000.
Marmur and Doty, J Mol. Biol. 5:109, 1962.

1. A method for generating a population of single stranded DNA molecules representative of a DNA sample, said method comprising:
(i) digesting the DNA sample with a Type I restriction endonuclease to generate DNA fragments having a reverse strand and a forward strand, wherein each DNA fragment comprises a double stranded portion with either two 3′ overhang portions or two 5′ overhang portions;
(ii) subjecting the DNA fragments to melt conditions to produce a melt-generated mixture of forward and reverse single stranded DNA fragments;
(iii) contacting the mixture of forward and reverse single strand DNA fragments with a solid support having immobilized thereon the complement of either the (a) forward or (b) reverse strand and subjecting the mixture to hybridization conditions which competitively favor hybridization of either (a) the reverse single stranded DNA fragment to the complement of the reverse strand immobilized to the solid support compared to the forward single stranded DNA fragment in the melt-generated mixture, or (b) the forward single stranded DNA fragment to the complement of the forward strand immobilized to the solid support compared to the reverse single strand DNA fragment in the melt-generated mixture, so as to capture the forward or reverse single strand DNA fragment on the solid support; and
(iv) releasing the captured forward or reverse single strand DNA fragments from the complement immobilized to the solid support to generate a population of single stranded DNA molecules representative of the DNA sample.

2. The method of claim 1 wherein the generated population of single stranded DNA molecules are used as capture molecules when immobilized to a solid support.

3. The method of claim 1 wherein the generated population of single stranded DNA molecule are used as detection probes.

4. The method of claim 1 wherein the Type I restriction endonuclease is BaeI.

5. The single stranded DNA fragments generated by the method of claim 1 immobilized to a microsphere.

6. The method of claim 1, wherein the DNA sample is a genome.


SEQUENCE LISTING (8 sequences)

SEQ ID NO: 1 - DNA, 13 bp, Artificial Sequence
  Restriction enzyme recognition sequence
gaacnnnnnntcc

SEQ ID NO: 2 - DNA, 11 bp, Artificial Sequence
  Restriction enzyme recognition sequence
gagnnnnnctc

SEQ ID NO: 3 - DNA, 11 bp, Artificial Sequence
  Restriction enzyme recognition sequence
acnnnngtayc

SEQ ID NO: 4 - DNA, 11 bp, Artificial Sequence
  Restriction enzyme recognition sequence
aagnnnnnctt

SEQ ID NO: 5 - DNA, 11 bp, Artificial Sequence
  Restriction enzyme recognition sequence
gaynnnnnvtc

SEQ ID NO: 6 - DNA, 12 bp, Artificial Sequence
  Restriction enzyme recognition sequence
gaacnnnnnctc

SEQ ID NO: 7 - DNA, 13 bp, Artificial Sequence
  Restriction enzyme recognition sequence
gaacnnnnnntac

SEQ ID NO: 8 - DNA, 27 bp, Artificial Sequence
  Synthetic DNA
nnnnnnnnnnacnnngtaycnnnnnnn